(19) United States
(12) Patent Application Publication
(10) Pub. No.: US 2019/0262268 A1
(43) Pub. Date: Aug. 29, 2019

(54) ORAL MODIFIED RELEASE FORMULATIONS

(71) Applicant: RUBICON RESEARCH PRIVATE LIMITED, Mumbai (IN)

(72) Inventors: Pratibha Pilgaonkar, Mumbai (IN); Anilkumar Gandhi, Mumbai (IN); Paras Jain, Mumbai (IN)

(21) Appl. No.: 16/334,976

(22) PCT Filed: Sep. 20, 2017
(86) PCT No.: PCT/IN2017/050416

§ 371 Date: Mar. 20, 2019

(30) Foreign Application Priority Data
Sep. 20, 2016 (IN) 201621009773

(51) Int. Cl. A61K 9/10; A61K 31/09; A61K 31/155; A61K 31/55; A61K 45/06; A61K 9/00; A61K 9/16

(52) U.S. Cl. CPC A61K 9/10; A61K 31/09; A61K 31/155; A61K 31/55; A61K 9/1652; A61K 9/0095; A61K 9/1635; A61K 9/1623; A61K 45/06

(57) ABSTRACT

The present invention relates to oral modified release compositions comprising plurality of modified release matrix particles and at least one pharmaceutically acceptable excipient; wherein the modified release matrix particles comprise at least one active agent, at least one release controlling agent and at least one pharmaceutically acceptable excipient. Further, the modified release composition is in the form of a suspension, dry formulation for reconstitution or dispersible tablet. The present invention also relates to a process for the preparation of these oral modified release pharmaceutical formulations.

### FIELD OF THE INVENTION

The present invention relates to oral modified release pharmaceutical formulations comprising plurality of modified release matrix particles and at least one pharmaceutically acceptable excipient. Particularly, the modified release matrix particles comprise at least one active agent, at least one release controlling agent and at least one pharmaceutically acceptable excipient. Further, the modified release composition is in the form of a suspension, dry formulation for reconstitution or dispersible tablet. The present invention also relates to a process for the preparation of the oral modified release pharmaceutical formulations.

### BACKGROUND OF THE INVENTION

The modified release pharmaceutical formulations offer many advantages over conventional immediate release formulations such as desired therapeutic effect for long period of time, attenuation of adverse effects, and improved patient convenience.

The common dosage forms for modified release of active agents by oral route of administration are solid dosage forms such as sustained release tablets and capsules. In general, however liquid dosage forms offer advantages over conventional solid dosage forms. Liquid dosage forms are palatable, suitable for high dose actives, and contrary to a fixed tablet dose provide better dose control. Liquid formulations or formulations for reconstitution before administration are easy to measure and ingest and are convenient for administration of drugs to pediatric or geriatric populations or to patients with dysphagia. Development of modified release liquid dosage forms is particularly challenging, since the formulation should maintain its modified release characteristics over the shelf life in a liquid environment and yet provide the desired release profile upon administration. The leaching of the drug from the modified release suspended phase into the surrounding aqueous media during storage needs to be particularly controlled.

Ion-exchange resin-drug complexes have been used to formulate pharmaceutical products of acidic and basic drugs. U.S. Pat. No. 2,990,332 discusses the use of an ion exchange resin to form a complex with ionic drugs and thereby delay the drug release from such complexes. However the degree of control over drug release from drug-resin complexes was found to be unsatisfactory since for many drugs only a relatively short delay in drug release was obtained. Attempts were therefore made to coat drug-resin complexes so as to provide a further diffusion barrier to drug release and thereby offer greater control over the drug release profile (U.S. Pat. Nos. 3,138,525, 3,499,960). However, the diffusion barrier coating of such coated drug-resin complexes when suspended in liquid dosage forms or upon contact with gastrointestinal fluids has a tendency to rupture due to the swelling of the resin resulting in loss of control of drug release and dose dumping. Further, leaching of the drug through the polymer coating into the liquid suspension base on storage though not significant with the ion exchange resin based systems, is yet observed at times based on the ionic strength of the suspension base used, and impacts the stability of the formulation and the desired controlled release of the drug when it enters the gastrointestinal tract. Furthermore, using ion exchange resins can be restrictive in terms of the variety of active principles that can be employed, drug loadings feasible and the variability in release profiles observed at times because the release rate of drug from drug-ion exchange resin is proportional to the concentration of the ions present in the area of administration and thereby on the surrounding gastrointestinal medium. Additionally, excessive coating levels using water permeable diffusion barrier generally applied over the drug-resin complexes for achieving effective release control makes the process time consuming, laborious and expensive.

Alternative strategies excluding ion-exchange resins have therefore been attempted for delivering active agents from a liquid formulation or a dry formulation for reconstitution. U.S. Pat. No. 5,296,236 discusses controlled release pharmaceutical dosage form, including: (a) microgranules of a pharmaceutical and an excipient; (b) a plurality of polymeric lipidic and wax-like coatings applied to the microgranules, the coated microgranules having dimensions which allow suspension of the coated microgranules in a liquid administration vehicle; and (c) a liquid administration vehicle for the coated microgranules, the vehicle including an effective amount of the pharmaceutical in a form immediately available upon ingestion. PCT Publication WO2011107855 discloses stable sustained release liquid oral suspension dosage form comprising: sustained release pellets comprising inert pellets, surrounded by seal coating, drug layer comprising pharmaceutically active ingredient with one or more pharmaceutically acceptable excipients surrounding said seal coated inert pellets, coating layer comprising rate controlling polymer surrounding said drug layer. Such sustained release pellets are suspended with suitable suspending agent, in addition to other pharmaceutically acceptable excipients in a suspending media at a suitable pH. U.S. Pat. No. 7,906,145 discloses an oral suspension comprising a plurality of microcapsules and an aqueous liquid phase that is saturated or becomes saturated with active principle on contact with the microcapsules. Each microcapsule comprises a core comprising at least one active principle, and a film coating that is applied to the core, controls the modified release of the active principle in gastrointestinal tract fluids, and comprises: (1) at least one film-forming polymer insoluble in gastrointestinal tract fluids; (2) at least one nitrogen-containing polymer; (3) at least one plasticizer; and (4) at least one surfactant or lubricant. PT Publication WO2015166473 discusses an extended release suspension composition comprising: (a) multiple coated cores having a core comprising an active ingredient; and a coating layer over said core comprising one or more release-controlling polymers; and (b) a suspension base, wherein the suspension base generates a hypertonic condition such that there is no substantial change in the in-vitro dissolution release profile of the extended release suspension compositions upon storage for at least seven days.

The above formulations incorporating drug containing coated dispersed phase, however involves the use of complex, time consuming coating processes for applying multiple coating layers. This makes the final product less economical and more laborious to prepare. Further the release of drug from the core or maintenance of the modified-release profile of the coated dispersed particles on storage in the aqueous phase depends on the composition and properties of the coating e.g. thickness and permeability of the coating and also on the strategies employed to avoid the leaching of the active into the suspension base over the shelf life of the liquid product. These coated drug particles are prone to dose dumping due to possible rupture of coat or increase in its permeability in the gastrointestinal tract (agitations, pH effects) resulting in loss of controlled release properties and high pharmacokinetic variability. Furthermore, swelling type of coatings if employed may also add to the variability in the desired release profile of the formulations.

A need therefore exists in the art to formulate modified release liquid compositions in the form of suspensions or compositions for reconstitution before administration that are based on simple and economical technology and provide the desired modified release throughout the shelf life of the composition. The present inventors have surprisingly found that an oral modified release pharmaceutical formulation comprising plurality of modified release uncoated matrix particles that are non-swelling in nature can overcome the above mentioned drawbacks associated with coated dispersed phase type systems or ion exchange resin based systems.

The compositions of the present invention can provide substantially similar in-vitro modified release profile of the active agent over the shelf life of the compositions. Such an in-vitro release then ensures a steady plasma concentration with no fluctuations throughout the shelf life of the compositions. Further the non-swelling nature of the modified release matrix also eliminates any associated inter-subject variability. The modified release compositions of the present invention are palatable, and easy to administer, thereby leading to enhanced patient compliance. Further, said modified release compositions can be used to deliver different dose ranges of the active agent and can also provide better dose flexibility depending on the age and body weight of the patient. Also, the modified release compositions of the present invention are stable, easy to commercially manufacture, and reproducible.

Particularly, oral modified release pharmaceutical formulation of the present invention comprise plurality of modified release matrix particles comprising at least one active agent, at least one release controlling agent and at least one pharmaceutically acceptable excipient dispersed in a saturated suspension base. Further, the oral modified release composition of the present invention comprise plurality of modified release matrix particles and at least one pharmaceutically acceptable excipient; wherein the modified release matrix particles comprise at least one active agent, at least one release controlling agent and at least one pharmaceutically acceptable excipient, and the modified release composition is in the form of a suspension, dry formulation for reconstitution or dispersible tablet. The formulation of the present invention overcomes the drawbacks such as leaching out of active agent, rupturing of the coat and dose dumping and offers desired modified release profile over the shelf life of the product.

### SUMMARY OF THE INVENTION

The present invention relates to oral modified release compositions comprising plurality of modified release matrix particles and at least one pharmaceutically acceptable excipient; wherein the modified release matrix particles comprise at least one active agent, at least one release controlling agent and at least one pharmaceutically acceptable excipient. Further, the modified release composition is in the form of a suspension, dry formulation for reconstitution or dispersible tablet. The present invention also relates to a process for the preparation of these oral modified release pharmaceutical formulations.

### DETAILED DESCRIPTION OF THE INVENTION

The present invention relates to oral modified release pharmaceutical formulations comprising plurality of modified release matrix particles. Particularly the modified release matrix particles comprise at least one active agent, at least one release controlling agent and at least one pharmaceutically acceptable excipient.

The term “composition” or “formulation” or “dosage form” has been employed interchangeably for the purpose of the present invention and mean that it is a pharmaceutical formulation which is suitable for administration to a patient. For the purpose of the present invention, the terms “controlled release” or “sustained release” or “extended release” or “modified release” or “prolonged release” have been used interchangeably and mean broadly that the active agent is released at a predetermined rate that is different or slower than immediate release. The term “active agent/s”, as employed herein refers to any suitable drug for which modified release is desired. In general all, including, but not limited to, acidic, basic, or amphoteric drugs, especially those having short biological half-lives such as in the order of up to about 12 hours are potential candidates in the compositions of the present invention. Active agents that exhibit a tendency to cause severe side effects when administered frequently in immediate release formulations are also potential candidates in the composition of the present invention.

Active agents that can be included in the present invention include, one or more psychostimulants, such as, but not limited to, amphetamine, amphetaminil, atomoxetine, dexmethylphenidate, dextroamphetamine, dextromethamphetamine, fencamfamine, fenethylline, lisdexamfetamine, methylphenidate, mesocarb, pemoline, pipradrol, prolintane and the like; or combinations thereof; antihistamines such as, but not limited to, dimenhydrinate, diphenhydramine, chlorpheniramine, brompheniramine, dexchlorpheniramine, hydroxyzine, dexbrompheniramine, fexofenadine, terfenadine, cetirizine, levocetirizine and the like; or combinations thereof; expectorants or mucolytics such as, but not limited to, ambroxol, bromhexine, carbocisteine, domiodol, guaifenesin and the like; or combinations thereof; anti-tussive agents such as, but not limited to, codeine, dextromethorphan, hydrocodone and the like; or combinations thereof; serotonin and norepinephrine reuptake inhibitor such as, but not limited to, clovoxamine, desvenlafaxine, duloxetine, levomilnacipran, eclanamine, milnacipran, sibutramine, venlafaxine, alaproclate, citalopram, escitalopram, femoxetine, fluoxetine, fluvoxamine, indalpine, ifoxetine litoxetine, omiloxetine, panuramine, paroxetine, pirandamine, seproxetine, sertraline zimelidine, and the like; or combinations thereof; sympatholytics such as, but not limited to, clonidine, guanfacine, methyldopa and the like; or combinations thereof; antipsychotics such as, but not limited to, iloperidone, ocaperidone, paliperidone, risperidone, lurasidone, perospirone, revospirone, tiospirone, ziprasidone and the like; or combinations thereof; antimuscarinics and urinary antispasmodics such as, but not limited to, darifenacin, emepronium, fesoterodine, flavoxate, imidafenacin, meladrazine, mirabegron, oxybutynin, propiverine, solifenacin, terodiline, tolterodine, trospium chloride and the like; or combinations thereof; PDE5 inhibitors such as, but not limited to, acetildenafil, aildenafil, avanafil, icariin, lodenafil, mirodenafil, nitrosoprodenafil, sildenafil, sulfoaildenafil, tadalafil, udenafil, vardenafil and the like; or combinations thereof; anti-Alzheimer's agent such as, but not limited to, memantine, neramexane (1,3,3,5,5-pentamethylcyclohexan-1-amine), donepezil, tacrine, rivastigmine, galantamine, physostigmine, neostigmine, Huperzine A, icopezil (CP-118954, 5,7-dihydro-3-[2-[1-(phenylmethyl)-4-piperidinyl]ethyl]-6H-pyrrolo-[4,5-f]-1,2-benzisoxazol-6-onemaleate), ER-127528 (4-[(5,6-dimethoxy-2-fluoro-1-indanon)-2-yl]methyl-1-(3-fluorobenzyl) piperidine hydrochloride), zanapezil (TAK-147; 3-[1-(phenylmethyl)piperidin-4-yl]-1-(2,3,4,5-tetrahydro-1H-1-benzazepin-8-yl)-1-propane fumarate), metrifonate (T-588; (−)-R-α-[[2-(dimethylamino)ethoxy]methyl]benzo[b] thiophene-5-methanol hydrochloride), FK-960 (N-(4-acetyl-1-piperazinyl)-p-fluorobenzamide-hydrate), TCH-346 (N-methyl-N-2-pyropinyldibenz[b,f] oxepine-10-methanamine), SDZ-220-581 ((S)-α-amino-5-(phosphonomethyl)-[1,1′-biphenyl]-3-propionic acid), and the like or combinations thereof, tarenflurbil, tramiprosate, clioquinol and the like; or combinations thereof; analgesics such as, but not limited to, aspirin, codeine, morphine, dihydromorphone, oxycodone, hydrocodone and the like or combinations thereof; decongestants such as, but not limited to, phenylephrine, pseudoephedrine and the like or combinations thereof; analeptic agents; anesthetic agents; anti-asthmatics such as, but not limited to, theophylline and the like or combinations thereof; anti-arthritic agents; anti-cancer agents; anti-cholinergic agents; anti-convulsant agents such as, but not limited to, phenobarbital sodium, phenytoin sodium, valproate sodium barbiturates, amylobarbitone sodium, butabarbital sodium, secobarbital sodium and the like or combinations thereof; anti-depressant agents; antidiabetics such as metformin; anti-helminthic agents; anti-diarrheal agents; anti-epileptics such as, but not limited to, phenytoin, meprobamate, nitrezepam, oxcarbazepine and the like or combinations thereof; anti-hyperlipidemic agents; antihypertensives such as, but not limited to, clonidine, methyldopa; captopril and the like or combinations thereof; antihypotensives such as, but not limited to, propranolol, clonidine and the like or combinations thereof; anti-infective agents; anti-inflammatory agents; non-steroidal anti-inflammatory agents such as, but not limited to, naproxen, diclofenac, indomethacin, ibuprofen, sulindac, meclofenamate sodium, tolmetin sodium and the like or combinations thereof; anti-emetics such as, but not limited to, metoclopramide and the like or combinations thereof; anti-migraine agents; anti-neoplastic agents; anti-tubercular agents; antibiotics such as, but not limited to, tetracyclines and the like or combinations thereof; antacids; antiulcer agents; anti-Parkinsonism drugs; anti-pruritic agents; antipsychotic agents; anti-pyretic agents; anti-spasmodics such as, but not limited, atropine, scopolamine and the like or combinations thereof; anti-viral agents; anxiolytic agents; appetite suppressants; attention deficit hyperactivity disorders; cardiovascular agents including, but not limited to, calcium channel blockers, antianginal agents; central nervous system agents; beta-blockers; antiarrhythmic agents; bronchodilators such as, but not limited to, albuterol; central nervous system stimulants; diuretics such as, but not limited to, ethacrynic acid, bendrofluazide and the like or combinations thereof; genetic materials; hormonolytics; hypnotics; hypercalcemics; hypoglycemic agents; immunosuppressive agents; beta-agonists; narcotic antagonists; nicotine; nutritional agents; parasympatholytics; peptide drugs; antihemorrhoidals; psychostimulants; psychotropics; mucolytics; sedatives; laxatives; vitamins; sialagogues, steroids; sympathomimetics; tranquilizers; vasodilators such as, but not limited to, nifedipine, papaverine, diltiazem, nicardirine and the like; or combinations thereof. In one embodiment, combinations of one or more active agents listed above may also be employed in the compositions of the present invention. The active agent/s employed in the compositions of the present invention may be in the form of free base or acid or pharmaceutically acceptable salts, prodrugs, active metabolites, polymorphs, solvates, hydrates, enantiomers, optical isomers, tautomers or racemic mixtures thereof.

Pharmaceutically effective amount of active agent is employed in the composition of the present invention. The term “effective amount” refers to an amount effective to achieve desired preventive, therapeutic and/or beneficial effect. In one embodiment the amount of active agent in the modified release matrix particles can vary from about 0.01 weight % to about 85 weight %, based on the total weight of the particles. In another embodiment the amount of active agent in the modified release matrix particles can vary from about 0.02 weight % to about 80 weight %, based on the total weight of the particles. In still another embodiment, the amount of active agent in the modified release matrix particles can vary from about 0.05 weight % to about 75 weight %, based on the total weight of the particles.

In one embodiment, the formulation of the present invention can be used to deliver different dose ranges of the active agent. In another embodiment, the formulation of the present invention can be used to deliver high dose of active agent. In a further embodiment, the formulation of the present invention can be used to deliver low dose of active agent. In another embodiment, the formulation of the present invention can be used to deliver an active having a high dose. In a further embodiment, the formulation of the present invention can be used to deliver an active having a low dose. In a further embodiment, the formulation of the present invention can be used to deliver active agents having any solubility. In another embodiment, the formulation of the present invention can be used to deliver active agents with low solubility. In a further embodiment, the formulation of the present invention can be used to deliver active agents with high solubility. In a further embodiment, active agents having solubility falling in any solubility range can be delivered by the formulations of the present invention. In one embodiment, active agents having high solubility and high dose can be delivered by the formulations of the present invention. In another embodiment, active agents having high solubility and low dose can be delivered by the formulations of the present invention. In a further embodiment, active agents having low solubility and high dose can be delivered by the formulations of the present invention. In one embodiment, active agents having low solubility and low dose can be delivered by the formulations of the present invention.

The modified release matrix particles of the present invention further comprise at least one release controlling agent. In a further embodiment, at least one active agent is dispersed in at least one release controlling agent. In another embodiment, at least one active agent is present in the matrix of at least one release controlling agent. In a further embodiment, the release controlling agent controls the release of the active agent through the modified release matrix particle.

In one embodiment, the release controlling agent employed in the modified release matrix particles of the present invention is non-swelling in nature. Without being bound to any theory it is believed that use of swelling type of release controlling agents generally cause variabilities in drug release and absorption. Particularly with extended release suspension formulations or reconstituted suspensions where matrix particles remain dispersed in the aqueous external phase, continuous water absorption and swelling by the swelling type rate controlling agents gradually results in the formation of very thin matrix and eventually loss of integrity of the particles and their modified release properties. The use of non-swelling type of release controlling agent limits the swelling of the modified release matrix particles and thereby reduces the variabilities associated with the use of such swelling excipients. The modified release particles of the present invention therefore deliver the active agent at a predetermined rate.

In one embodiment, the release controlling agent used in the present invention may be polymeric or non-polymeric or the mixtures thereof. In another embodiment, the release controlling agent employed in the present invention may be non-swelling in nature. In one embodiment, the release controlling agent employed is a non-swelling release controlling agent. In a further embodiment, the release controlling agent employed in the present invention may be non-swelling polymeric or non-swelling non-polymeric release controlling agent or a mixture thereof. The term “non-swelling” as employed in the present invention means that the release controlling agent/s or the modified release matrix particles does not exhibit swelling of more than 50% of their initial volume within one hour after introduction in water. In a further embodiment, the term “non-swelling” as employed in the present invention means that the release controlling agent/s or the modified release matrix particles does not exhibit swelling of more than 30% of their initial volume within one hour after introduction in water. In yet another embodiment, the term “non-swelling” as employed in the present invention means that the release controlling agent/s or the modified release matrix particles does not exhibit swelling of more than 20% of their initial volume within one hour after introduction in water. In a further embodiment, the polymeric release controlling agent may be pH-dependent or pH-independent in nature or combinations of pH dependent or pH independent polymeric release controlling agents may be employed.

In another embodiment, the polymeric release controlling agents may be selected from, but are not limited to, cellulose derivatives, acrylic acid derivatives, maleic acid derivatives, polymers and copolymers, vinyl derivatives, polymers and copolymers and the like or combinations thereof. Cellulose derivatives that may be employed in the particles of the present invention include, but are not limited to, ethyl cellulose, hydroxypropylmethylcellulose acetate succinate, hydroxypropylmethylcellulose phthalate, hydroxymethylethylcellulose phthalate, cellulose acetate phthalate, cellulose acetate succinate, cellulose acetate maleate, cellulose acetate trimelliate, cellulose benzoate phthalate, cellulose propionate phthalate, methylcellulose phthalate, carboxymethylethylcellulose, ethylhydroxyethylcellulose phthalate and the like or combinations thereof. Acrylic acid derivatives include, but are not limited to, styrene/acrylic acid copolymer, methyl acrylate/methacrylic acid copolymer, butyl acrylate/styrene/acrylic acid copolymer, methacrylic acid polymers and copolymers, polyacrylates, methacrylate polymers and copolymers such as, but not limited to, a) copolymer formed from monomers selected from methacrylic acid, methacrylic acid esters, acrylic acid and acrylic acid esters b) copolymer formed from monomers selected from butyl methacrylate, (2-dimethylaminoethyl)methacrylate and methyl methacrylate c) copolymer formed from monomers selected from ethyl acrylate, methyl methacrylate and trimethylammonioethyl methacrylate chloride or d) copolymers of acrylate and methacrylates with/without quarternary ammonium group in combination with sodium carboxymethylcellulose, e.g. those available from Rohm GmbH under the trademark Eudragit® like Eudragit® EPO (dimethylaminoethyl methacrylate copolymer; basic butylated methacrylate copolymer), Eudragit® RL and RS (trimethylammonioethyl methacrylate copolymer), Eudragit® NE30D and Eudragit® NE400 (ethylacrylate methymethacrylate copolymer), Eudragit® L 100 and Eudragit® S (methacrylic acid-methyl methacrylate copolymer), Eudragit® L100-55 (methacrylic acid-ethyl acrylate copolymer); or the like or any combinations thereof. Maleic acid derivatives, polymers and copolymers that may be employed in the particles of the present invention include, but are not limited to, vinylacetate/maleic acid anhydride copolymer, styrene/maleic acid anhydride copolymer, styrene/maleic acid monoester copolymer, vinylmethylether/maleic acid anhydride copolymer, ethylene/maleic acid anhydride copolymer, vinylbutylether/maleic acid anhydride copolymer, acrylonitrile/methyl acrylate/maleic acid anhydride copolymer, butyl acrylate/styrene maleic acid anhydride copolymer and the like. Vinyl derivatives, polymers and copolymers include, but are not limited to, polyvinylacetate, copolymers of vinyl pyrrolidone, polyvinyl alcohol, copolymers of polyvinyl alcohol, mixture of polyvinyl acetate and polyvinylpyrrolidone (e.g. Kollidon® SR), polyvinyl alcohol phthalate, polyvinylacetal phthalate, polyvinyl butylate phthalate, polyvinylacetoacetal phthalate, or combinations thereof.

In one embodiment, the polymeric release controlling agent employed in the modified release matrix particles of the present invention is mixture of polyvinyl acetate and polyvinylpyrrolidone, such as, but not limited to, Kollidon® SR (which is a mixture of 8 parts w/w of polyvinyl acetate and 2 parts w/w of polyvinyl pyrrolidone). In a further embodiment, the polymeric release controlling agent employed is Kollidon® SR. In another embodiment, a combination of Kollidon® SR and another polymeric or non-polymeric release controlling agent is employed to prepare modified release matrix particles.

In a further embodiment, the non-polymeric release controlling agents that may be employed in the particles of the present invention include, but are not limited to, sucrose fatty acid esters, glyceryl fatty acid esters, fatty acid alkyl esters, propylene glycol esters of fatty acids, sorbitan fatty acid esters, or combinations thereof. Sucrose fatty acid esters, generally called sugar esters are nonionic surfactants consisting of sucrose as hydrophilic group and fatty acid as lipophilic group. As sucrose has a total of 8 hydroxyl groups, compounds ranging from sucrose mono- to octa-fatty acid esters can be produced. Sucrose fatty acid esters that may be employed in the particles of the present invention include, but are not limited to sucrose mono- to octa-fatty acid esters and various combinations and proportions thereof. Suitable sucrose fatty acid esters that may be employed include, but are not limited to, sucrose stearate, sucrose distearate, sucrose polystearate, sucrose palmitate, sucrose monopalmitate, sucrose dipalmitate, sucrose oleate, sucrose monooleate, sucrose dioleate, sucrose laurate, sucrose monolaurate, sucrose dilaurate, sucrose behenate, sucrose erucate, sucrose caprate, sucrose monocaprate, sucrose dicaprate, sucrose caprylate, sucrose monocaprylate, sucrose dicaprylate, sucrose myristate, sucrose monomyristate, sucrose dimyristate, sucrose linolenate, sucrose monolinolenate, sucrose dilinolenate, sucrose esters of mixed fatty acids, sucrose oligoesters, and mixtures thereof. In one embodiment, sucrose esters commercially available under the trademark SURFHOPE™ SE PHARMA from Mitsubishi Kagaku, Tokyo, or under the trademark SUCROESTER™ from Gattefosse and in many grades depending upon the fatty acid employed, the proportion of the mono- to octa-esters present and HLB (hydrophilic lipophilic balance) value may be employed in the compositions of the present invention. In one embodiment, the sucrose fatty acid ester having HLB from about 1 to about 16 may be employed in the particles of the present invention. In another embodiment, sucrose fatty acid ester having HLB from about 1 to about 10 may be employed in the particles of the present invention. In a further embodiment, sucrose fatty acid ester having HLB from about 1 to 5 may be employed in the particles of the present invention. Glyceryl fatty acid esters that may be employed in the present invention include, but are not limited to, mono-, di- and triglycerides. Suitable glyceryl fatty acid esters include, but are not limited to, glyceryl behenate; glyceryl monostearate, glyceryl tripalmitate, glyceryl monopalmitate, glyceryl distearate, glyceryl tristearate glyceryl palmitostearate, glyceryl dipalmitate, glyceryl monoleate, glyceryl monolaurate, glyceryl dilaurate, glyceryl trilaurate, glyceryl didocosanoate glyceryl tridocosanoate, glyceryl monodocosanoate, glyceryl monocaprote, glyceryl dicaproate, glyceryl tricaproate, glyceryl monomyristate, giyberyl dimyistate, glyceryl trimyristate, glyceryl monodecenoate, glyceryl didecenoate, glyceryl tridecenoate; GL YCOWAX-932; lauroyl macrogol-32 glycerides; stearoyl macrogol-32 glyceride; fatty acid esters such as those having a fatty acid chain length of about C₁₀-C₄₀; glyceryl monoarachidonate, glyceryl monolinoleate, glyceryl monolinolenate, glyceryl monopalmitoleate, glyceryl monocaprylate, glyceryl monococoate, glyceryl monocollagenate, glyceryl monoerucate, glyceryl monohydroxystearate, glyceryl monoisopalmitate, glyceryl monolinoleate, glyceryl monolinolenate, glyceryl monomyristate, glyceryl monopentadecanoate, glyceryl monopolyacrylate, glyceryl monotallowate, glyceryl monothiopropionate, glyceryl monocundecylenate or mixtures thereof. Suitable fatty acid alkyl esters that may be employed in the compositions of the present invention include, but are not limited to, isopropyl monoarachidonate, isopropyl monolaurate, isopropyl monolinoleate, isopropyl monolinolenate, isopropyl monomyristate, isopropyl monopalmitoleate, isopropyl monooleate, and isopropyl monostearate; methyl monoarachidonate, methyl monolaurate, methyl monolinoleate, methyl monolinolenate, methyl monomyristate, methyl monopalmitoleate, methyl monooleate, methyl monostearate or mixtures thereof. Suitable propylene glycol esters of fatty acid that may be employed include, but are not limited to, propylene glycol monoarachidonate, propylene glycol monolaurate, propylene glycol monolinoleate, propylene glycol monolinolenate, propylene glycol monomyristate, propylene glycol monopalmitoleate, propylene glycol monooleate, propylene glycol monostearate or a combination thereof. Suitable sorbitan fatty acid esters that may be employed in the compositions of the present invention include, but are not limited to, sorbitan monolaurate, sorbitan monopalmitate, sorbitan monooleate, sorbitan monostearate, sorbitan tristearate, sorbitan trioleate, sorbitan sesquioleate, sorbitan tistearate, sorbitan monoisostearate, sorbitan sesquistearate, sorbitan caprylate, sorbitan cocoate, sorbitan diisostearate, sorbitan dioleate, sorbitan distearate, sorbitan isostearate, sorbitan olivate, sorbitan sesquiisostearate, sorbitan sesquistearate, and sorbitan triisostearate, and the like or combinations thereof.

In one embodiment, the non-polymeric release controlling agent that may be employed in the modified release matrix particles of the present invention is sucrose fatty acid ester. In another embodiment, the non-polymeric release controlling agent that may be employed is combination of sucrose fatty acid ester and at least one other polymeric or non-polymeric release controlling agent or any combination thereof.

The amount of release controlling agent used in the formulation may vary depending upon the active agent employed and the degree of modified release desired. In one embodiment, the release controlling agent is present in the modified release matrix particles of the present invention in an amount from about 5% to about 95% by weight of the particles. In another embodiment, the release controlling agent is present in the modified release matrix particles of the present invention in an amount from about 10% to about 90% by weight of the particles. In a further embodiment, the release controlling agent is present in the modified release particles of the present invention is in an amount from about 15% to about 85% by weight of the particles.

The modified release particles comprising at least one active agent and at least one release controlling agent further comprise at least one pharmaceutically acceptable excipients, such as, but not limited to, diluents, binders, flavors, sweeteners, colorants, glidants, lubricants and the like or any combinations thereof. Suitable diluents that may be employed include, but are not limited to, starch, microcrystalline cellulose, lactose, xylitol, mannitol, maltose, polyols, fructose, sorbitol, magnesium hydroxide, dicalcium phosphate, and the like or any combinations thereof. Suitable binders that may be employed include, but are not limited to, starch, pregelatinized starch, polyvinyl pyrrolidone, copovidone, cellulose derivatives, such as hydroxypropylmethyl cellulose, hydroxypropyl cellulose and carboxymethyl cellulose and their salts. Suitable lubricants that may be employed include, but are not limited to, magnesium stearate, calcium stearate, stearic acid, talc, and sodium stearyl fumarate. Suitable glidants that may be employed include but are not limited to, colloidal silica, silica gel, precipitated silica, or combinations thereof. In one embodiment, the modified release particles may also be suitably taste-masked to improve palatability. Suitable sweeteners, flavors, or colorants as listed herein under may be incorporated in the particles of the present invention.

In one embodiment, the modified release matrix particles of the present invention can be in the form of granules, pellets, microparticles, beads, spheroids, and the like or combinations thereof. In another embodiment, the modified release matrix particles of the present invention comprising at least one active agent, at least one release controlling agent and at least one pharmaceutically acceptable excipient, can be prepared according to methods such as, but not limited, wet granulation, dry granulation, roller compaction, extrusion-spheronization, melt granulation, physical mixing and compression or compaction or the combinations thereof.

In a further embodiment, the modified release matrix particles of the present invention may range in size from about 1 micrometer to about 1000 micrometers. In another embodiment, the modified release matrix particles of the present invention may range in size from about 1 micrometer to about 750 micrometers. In one embodiment, the modified release matrix particles of the present invention may range in size from about 5 micrometers to about 500 micrometers. In a further embodiment, the modified release matrix particles of the present invention are non-swelling in nature. In one embodiment, the modified release matrix particles of the present invention are not in the form of coated core. In another embodiment of the present invention the modified release matrix particles do not comprise any coating. In a further embodiment, the active agent is not present in the form of any coat in the modified release matrix particles.

In one embodiment, the modified release matrix particles of the present invention comprise at least one active agent uniformly dispersed in the matrix of at least one release controlling agent. In a further embodiment, the modified release matrix particles of the present invention comprise at least one active agent uniformly dispersed in the matrix of at least one release controlling agent and at least one pharmaceutically acceptable excipient. In another embodiment, the modified release matrix particles of the present invention may be present in the compositions of the present invention in an amount from about 5% to about 100% by weight of the composition. In a further embodiment, the modified release matrix particles of the present invention are incorporated in the modified release pharmaceutical formulations by any of the methods generally known to a person skilled in the art, especially depending on the final dosage form of the pharmaceutical composition.

Modified release pharmaceutical formulations of the present invention comprise plurality of modified release matrix particles. In one embodiment, different populations of modified release matrix particles exhibiting different release profiles may be incorporated in the modified release pharmaceutical formulations of the present invention. In a further embodiment, the modified release formulations of the present invention can comprise one or more populations of modified release matrix particles to provide the desired release profile of the active agent. In another embodiment, the modified release formulations of the present invention can comprise one or more populations of modified release matrix particles prepared using different release controlling agents or combinations thereof.

In a further embodiment, modified release pharmaceutical compositions of the present invention comprise multitude of modified release matrix particles and at least one pharmaceutically acceptable excipient. In one embodiment, the modified release compositions are for oral delivery. The compositions for oral delivery may be in any form, such as, but not limited to, liquid, solid or semisolid preparations and the like. Liquid preparations for oral administration may be in any form including, but not limited to, suspensions, syrups or the like. Solid preparations for oral administration may be in any form including, but not limited to, dispersible tablets, dry formulation for reconstitution, powder or granules for reconstitution and the like or any combinations thereof. In one embodiment the modified release preparation of the present invention is a suspension. In another embodiment, the modified release preparation of the present invention is a dry formulation for reconstitution. In one embodiment, the modified release preparation of the present invention is a dry formulation for reconstitution in the form of, but not limited to, powder, granules and the like or combinations thereof. In a further embodiment, the modified release preparation of the present invention is a dispersible tablet.

In one embodiment, the modified release preparation of the present invention is a suspension comprising plurality of modified release matrix particles dispersed in a suspension base. In another embodiment, the suspension base employed for dispersing the modified release matrix particles of the present invention is saturated with the active agent. In a further embodiment, the suspension base saturated with the active agent suppresses the diffusion of the active out from the modified release matrix particles. In another embodiment, the release profile of the modified release suspension formulation of the present invention remains unchanged over the shelf life of the formulation. In one embodiment, the leaching of the active from the modified release matrix particles is prevented over the shelf life of the composition. In one embodiment, modified release formulation of the present invention in the form of suspension is prepared by dispersing the modified release matrix particles in suspension base. In a further embodiment, the oral modified release composition of the present invention in the form of a suspension comprises the modified release matrix particles dispersed in a suspension base.

In a further embodiment, the modified release preparation of the present invention is a dry formulation for reconstitution comprising plurality of modified release matrix particles. In another embodiment, the dry formulation for reconstitution is dispersed in an aqueous medium and the reconstituted suspension is administered over a period of few days. In one embodiment, the dry formulation on reconstitution saturates the aqueous medium to create a saturated suspension base. In a further embodiment, the dry formulation for reconstitution has a component of active agent along with the modified release matrix particles that saturates the aqueous medium. In one embodiment, the dry formulation for reconstitution in addition to the modified release matrix particles comprises immediate release particles comprising the active agent which is being delivered in modified release manner, in an amount sufficient to saturate the aqueous medium and at least one pharmaceutically acceptable excipient. In a further embodiment, these immediate release particles saturate the aqueous medium on one hand to reduce leaching of the active from the modified release particles while on the other hand generate necessary viscosity in the aqueous medium to ensure uniform dispersion and dosing of the active. In another embodiment the dry formulation for reconstitution in addition to the modified release matrix particles comprises suspension modifying particles comprising at least one pharmaceutically acceptable excipient that generates necessary viscosity in the aqueous medium to ensure uniform dispersion and dosing of the active. In another embodiment, the release profile of the modified release reconstituted suspension formulation of the present invention remains unchanged over the shelf life of the formulation. In one embodiment, the leaching of the active from the modified release matrix particles is prevented over the shelf life of the composition. In another embodiment, the oral modified release composition in the form of dry formulation for reconstitution in addition to the modified release matrix particles, further comprises immediate release particles of the active agent and/or suspension modifying granules. In a further embodiment, the oral modified release composition in the form of a dry formulation for reconstitution, on reconstitution generates a suspension base saturated with the active agent wherein the modified release matrix particles are dispersed.

In a further embodiment, the modified release pharmaceutical formulations of the present invention based on use or generation of saturated suspension base, may include into the suspension or in the dry formulation for reconstitution, at least one agent for modifying the solubility of the active agent such as but not limited to glycerol, sugars, salts and the like or combinations thereof. In a further embodiment, the agents for modifying the solubility of the active agent include, but are not limited to, carbohydrates such as sucrose, xylitol, mannitol, sorbitol, arabinose, ribose, xylose, glucose, fructose, mannose, galactose, maltose, lactose, dextrose and raffinose; water-soluble salts of organic acids such as sodium citrate, sodium acetate, potassium acetate, magnesium succinate, sodium benzoate, and sodium ascorbate; water-soluble salts of inorganic acids such as magnesium chloride, sodium chloride, magnesium sulfate, potassium sulfate, sodium hydrogen phosphate, lithium chloride, potassium chloride, lithium hydrogen phosphate, potassium hydrogen phosphate, lithium dihydrogen phosphate, sodium dihydrogen phosphate, potassium dihydrogen phosphate, and sodium phosphate tribasic; water-soluble amino acids such as glycine, methionine, leucine, alanine; urea or its derivatives; propylene glycol; glycerin; polyethylene oxide; xanthan gum; hydroxypropylmethyl cellulose; or mixtures thereof.

In another embodiment, the formulations of the present invention minimize the leaching of the active ingredient from the modified release particles into the suspension base and thus providing substantially similar in-vitro modified release of the active ingredient throughout the shelf life of the compositions. The term “minimize leaching” as employed in the present invention means the leaching of the active is not more than 20% by weight of the active incorporated in the modified release matrix particles of the present invention. In another embodiment, the term “minimize leaching” as employed in the present invention means the leaching of the active is not more than 15% by weight of the active incorporated in the modified release matrix particles of the present invention. In a further embodiment, the term “minimize leaching” as employed in the present invention means the leaching of the active is not more than 10% by weight of the active incorporated in the modified release matrix particles of the present invention.

In one embodiment, the in-vitro dissolution release profile of the extended release suspension compositions of the present invention upon storage for at least three months remains substantially similar to initial in-vitro dissolution release profile. In a further embodiment, the in-vitro dissolution release profile of the extended release suspension compositions of the present invention upon storage for at least six months remains substantially similar to initial in-vitro dissolution release profile obtained. In another embodiment, the in-vitro dissolution release profile of the extended release suspension compositions of the present invention remains substantially similar to initial in-vitro dissolution release profile over the shelf life of the compositions.

In a further embodiment, the pH of the modified release suspension composition of present invention may be acidic or neutral or in the pH range of 2 to 10 depending on the stability, solubility of the active ingredient in the aqueous medium, the release profile and the site of release of the active agent. In one embodiment, the pH of the formulation of present invention after reconstitution may be maintained as acidic or neutral or in the pH range of 2 to 10 depending on the active agent, its properties and desired release profile.

In another embodiment, the formulation of the present invention is in the form of dispersible tablet composition comprising plurality of modified release matrix particles. In a further embodiment, the dispersible tablet on dispersion in an aqueous medium may not require saturation of the liquid phase. In a further embodiment, the formulation of the present invention in the form of dispersible tablet may be prepared by methods known in the art, such as, but not limited to, direct compression, wet granulation, dry granulation, and the like.

The modified release compositions of the present invention comprise at least one pharmaceutically acceptable excipient, depending on the final dosage form to be prepared, such as, but not limited to, binders, disintegrants, superdisintegrants, diluents, salivating agents, surfactants, flavors, sweeteners, colorants, souring agents, viscolizers, glidants, lubricants, solubilizers, stabilizers, suspending agents, preservatives, cosolvents, anti-caking agents, buffers and the like or any combinations thereof. Suitable disintegrants can be selected from, but not limiting to, crospovidone, calcium silicate and starch. Suitable superdisintegrants include, but are not limited to, natural, modified or pregelatinized starch, crospovidone, croscarmellose sodium, sodium starch glycolate, low-substituted hydroxypropyl cellulose. Examples of suitable binders include, but are not limited to, starch, pregelatinized starch, polyvinyl pyrrolidone, copovidone, cellulose derivatives, such as hydroxypropylmethyl cellulose, hydroxypropyl cellulose and carboxymethyl cellulose and their salts. Examples of suitable diluents include, but are not limited to, starch, microcrystalline cellulose, lactose, xylitol, mannitol, maltose, polyols, fructose, guar gum, sorbitol, magnesium hydroxide, dicalcium phosphate, coprocessed mannitol and calcium silicate and the like or any combinations thereof. Examples of lubricants include, but are not limited to, magnesium stearate, calcium stearate, stearic acid, talc, and sodium stearyl fumarate. Suitable glidants includes but are not limited to, colloidal silica, silica gel, precipitated silica, or combinations thereof. Suitable salivating agents include, but are not limited to, micronised polyethylene glycol, sodium chloride or precipitated micronised silica. Examples of solubilizers include, but are not limited to cetostearyl alcohol, cholesterol, diethanolamine, ethyl oleate, ethylene glycol palmitostearate, glycerin, glyceryl monostearate, isopropyl myristate, lecithin, medium-chain glyceride, monoethanolamine, oleic acid, propylene glycol, polyoxyethylene alkyl ether, polyoxyethylene castor oil glycoside, polyethylene sorbitan fatty acid ester, polyoxyethylene stearate, propylene glycol alginate, sorbitan fatty acid ester, stearic acid, sunflower oil, triethanolmine, or combinations thereof. Souring agents include, but are not limited to, monosodium fumarate and/or citric acid. The compositions of the present invention may also include stabilizers such as, but not limited to, those described above under drug-resin complexes. Suitable viscolizers include, but are not limited to, coprocessed microcrystalline cellulose such as but not limited to, Avicel RC591, Avicel CL-611, D-sorbitol solution, polyalkylene oxides such as, but not limited to polyethylene oxide; cellulose ethers such as, but not limited to hydroxyethyl cellulose, hydroxypropylcellulose, hydroxypropyl methyl cellulose, methyl cellulose, ethyl cellulose, sodium carboxy methylcellulose, calcium carboxymethyl cellulose, microcrystalline cellulose; gums such as but not limited to gum arabic alginates, agar, sodium alginate guar gum, locust bean, carrageenan, tara, gum arabic, tragacanth, pectin, xanthan, gellan, maltodextrin, galactomannan, pusstulan, laminarin, scleroglucan, gum arabic, inulin, karaya, whelan; polyols such as, but not limited to dipropylene glycol, polypropylene glycol, propylene glycol, polyethylene glycol (PEG), sorbitol and glycerol; carbopol, starch and starch-based polymers such as, but not limited to, pregelatinized starch, acrylic acid and methacrylic acid polymers, and esters thereof, maleic anhydride polymers; polymaleic acid; poly(acrylamides); poly(olefinic alcohol)s; poly(N-vinyl lactams); polyoxyethylated saccharides; polyoxazolines; polyvinylamines; polyvinylacetates; polyimines; povidone, vinylpyrrolidone/vinyl acetate copolymer and polyvinyl acetate, mixture of polyvinyl acetate and polyvinylpyrrolidone, chitin, cyclodextrin, gelatin, chitosan and the like or any mixtures thereof. Suitable surfactants include, but are not limited to, anionic, nonionic, cationic, and zwitterionic surfactants or a mixture thereof. The non-ionic surfactants employed in the composition may include, but are not limited to, ethoxylated fatty acid ester, ethoxylated fatty acid ethers, ethoxylated sorbitan ethers, ethoxylated alkyl-phenols, glycerol esters, glycerol sugar esters, polyoxyethyleneglycerol monolaurate, polyoxyethyleneglycerol monostearate, polyoxyethylene-20-cetyl stearate, polyoxyethylene-25-cetyl stearate, polyoxyethylene (25)-oxypropylene monostearate, polyoxyethylene-20-sorbitan monopalmitate, poly-oxyethylene-16-tert-octylphenol, polyoxyethylene-20-cetyl ether, polyethylene glycol(1000) monocetyl ether, ethoxylated castor oil, polyoxyethylene sorbitol-lanolin derivatives, polyoxyethylene(25) propylene glycol stearate, polyoxyethylenesorbitol esters, polyoxyethylene-20-sorbitan monopalmitate, polyoxyethylene-16-tert-octylphenol, polyoxyethylene-20-cetyl ether, glycyeryl undecylenate and Polysorbate 60, capmul (medium chain glyceride), peceol (glyceryl monooleate), glyceryl laurate and glyceryl caprylate (Capmul MCM), PEG sorbitan fatty acid esters like PEG-20 sorbitan monolaurate (Tween 20), PEG-20 sorbitan monostearate (Tween 60), PEG-20 sorbitan monooleate (Tween 80), sorbitan fatty acid esters like sorbitan monolaurate (Span 20), glyceryl stearate (Cithrol GMS) or the like and mixtures thereof. Suitable cationic surfactants include, but are not limited to, quaternary ammonium compounds, alkylamidoamines and quaternary ester compounds, distearyl dimethyl ammonium chloride, dimyristyl dimethyl ammonium chloride, dipalmityl dimethyl ammonium chloride or the like and mixtures thereof. Suitable anionic surfactants include, but are not limited to, fatty alcohol sulfates, alpha olefin sulfonates, sulfosuccinates, phosphate esters, carboxylates, sarcosinates, alkyl benzene sulfonates, alkyl sulfonates, olefin sulfonates, alkyl ethersulfonates, glycerol ethersulfonates, α-methyl estersulfonates, sulfonic fatty acids, alkyl sulfates, fatty alcohol ethersulfates, glycerol ethersulfates, mixed hydroxy ethersulfates, monoglyceride (ether)sulfates, fatty acid amide (ether)sulfates, sulfosuccinates, sulfosuccinamates, sulfotriglycerides, amide soaps, ether carboxylic acids, isethionates, sarcosinates, taurides, alkyl oligoglycoside sulfates, alkyl (ether)phosphates or the like and mixtures thereof. Suitable zwitterionic surfactants employed include, but are not limited to, N-alkyl-N,N-dimethyl ammonium glycinates, for example cocoalkyl dimethyl ammonium glycinate, N-acyl aminopropyl-N,N-dimethyl ammonium glycinates, cocoacyl aminoethyl hydroxyethyl carboxymethyl glycinate or the like and mixtures thereof. Further, the composition of the present invention may further comprise a preservative such as but not limited to methyl parahydroxybenzoate, propyl parahydroxybenzoate and sodium benzoate. Suitable cosolvent that may be used includes, but is not limited to, ethanol and polyhydric alcohols such as, but not limited to, glycerin, propylene glycol, low molecular weight polyethylene glycols, and mixtures thereof. Further anti-caking agents that may be optionally incorporated include, but are not limited to, colloidal silicon dioxide, tribasic calcium phosphate, powdered cellulose, magnesium trisilicate, starch, and mixtures thereof. Suitable sweetening agent includes, but is not limited to, aspartame, stevia extract, glycyrrhiza, saccharine, saccharine sodium, acesulfame, sucralose, dipotassium glycyrrhizinate, galactose, fructose, high fructose corn syrup, dextrose, sucrose, sugar, maltose, partially hydrolyzed starch, corn syrup solids, sorbitol, xylitol, mannitol and the like or mixtures thereof. The compositions may comprise one or more natural and/or artificial flavors such as, but not limited to, mint flavour, orange flavour, lemon flavors, strawberry aroma, vanilla flavour, raspberry aroma, cherry flavor, tutty frutty flavor, magnasweet 135, key lime flavor, grape flavor, trusil art 511815, and fruit extracts and the like. Suitable colorants include, but are not limited to, pigments and dyes such as FD&C Red, FD&C Yellow, FD&C Green, and FD&C Blue and the like or combinations thereof.

In one embodiment, the modified release formulation of the present invention in the form of suspension comprises (a) plurality of modified release matrix particles comprising at least one active agent, at least one sucrose fatty acid ester and at least one pharmaceutically acceptable excipient; and (b) suspension base. In another embodiment, the modified release formulation of the present invention in the form of suspension comprises (a) plurality of modified release matrix particles comprising at least one active agent, at least one sucrose fatty acid ester and at least one pharmaceutically acceptable excipient; and (b) suspension base saturated with the active agent present in the modified release matrix particles. In one embodiment, the modified release formulation of the present invention in the form of suspension comprises (a) plurality of modified release matrix particles comprising at least one active agent, mixture of polyvinyl pyrrolidone and polyvinyl acetate, and at least one pharmaceutically acceptable excipient; and (b) suspension base. In another embodiment, the modified release formulation of the present invention in the form of suspension comprises (a) plurality of modified release matrix particles comprising at least one active agent, mixture of polyvinyl pyrrolidone and polyvinyl acetate and at least one pharmaceutically acceptable excipient; and (b) suspension base saturated with the active agent present in the modified release matrix particles. In one embodiment, the modified release formulation of the present invention in the form of suspension comprises (a) plurality of modified release matrix particles comprising at least one active agent, Kollidon® SR, and at least one pharmaceutically acceptable excipient; and (b) suspension base. In another embodiment, the modified release formulation of the present invention in the form of suspension comprises (a) plurality of modified release matrix particles comprising at least one active agent, Kollidon® SR and at least one pharmaceutically acceptable excipient; and (b) suspension base saturated with the active agent present in the modified release matrix particles. In one embodiment, the modified release formulation of the present invention in the form of suspension comprises (a) plurality of modified release matrix particles comprising at least one active agent, at least one sucrose fatty acid ester and Kollidon® SR, and at least one pharmaceutically acceptable excipient; and (b) suspension base. In another embodiment, the modified release formulation of the present invention in the form of suspension comprises (a) plurality of modified release matrix particles comprising at least one active agent, at least one sucrose fatty acid ester and Kollidon® SR and at least one pharmaceutically acceptable excipient; and (b) suspension base saturated with the active agent present in the modified release matrix particles. In one embodiment, the suspension formulation or the reconstituted suspension or the dispersed dispersible tablet exhibit uniform dispersion or suspension of the modified release matrix particles to provide uniform dose of the active agent over a prolonged time period. In another embodiment, the formulations of the present invention are palatable easy to use formulations that exhibit improved patient compliance. In one embodiment, the modified release matrix particles of the present invention are designed such that the release profile of the active agent is not affected. The modified release compositions of the present invention can be readily formulated according to methods well known to those skilled in the art. Method of preparation of the compositions of the present invention depends on the final dosage form desired.

In a further embodiment, the compositions of the present invention provide modified release of the active agent in-vitro and in-vivo for up to about 24 hours. In one embodiment the compositions of the present invention provide modified release of the active agent in-vitro and in-vivo for up to about 12 hours. In another embodiment, the formulation of the present invention provides modified release of the active agent in-vitro for a period of about 4 hours or more. In a further embodiment, the formulation of the present invention provides modified release of the active agent in-vitro for a period of about 4 hours to about 24 hours. In the present invention, wide ranges of dissolution methodologies can be utilized for different active ingredients. These methodologies can be adopted to simulate in-vivo conditions by using different dissolution apparatuses, volume of media, pH of media ranging from 1.0 to 7.5, molarities, addition of surfactants, and or enzymes. In a further embodiment, the compositions of the present invention provide therapeutic effect for up to about 24 hours. In another embodiment, the compositions of the present invention provide therapeutic effect for up to about 12 hours. In one embodiment, the formulations of the present invention may be adapted for once or twice a day administration.

In one embodiment, the formulation of the present invention can adapted for delayed release of at least one active agent. In a further embodiment, the formulation of the present invention can be adapted for pulsed release of the active agent. In another embodiment, the formulation of the present invention can be adapted for colon-specific release of the active agent. In a further embodiment, the type of release is dependent of the active agent to be delivered and the desired therapeutic benefit.

In a further embodiment is provided use of the modified release compositions of the present invention for the prevention, treatment, management or mitigation of various disease conditions or disorders depending on the active agent employed.

In another embodiment, the present invention also relates to modified release compositions comprising at least one second active agent in addition to at least one active agent present in the compositions. Such a second active agent includes, but is not limited to, the list of active agents discussed above under active agents. In a further embodiment, the second active agent may be delivered in an immediate or modified release manner. In one embodiment of the present invention, the plurality of particles comprises same or different active agents. In another embodiment of the present invention, the active agents in the particles can be delivered at different release rate and profiles as desired.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings without departing from the essential scope thereof. Therefore, it is intended that the invention not be limited to the particular embodiment disclosed, but that the invention will include all embodiments falling within the scope thereof. Details of the present invention, including its objects and advantages, are provided in the non-limiting exemplary illustrations below.

### EXAMPLES

### Example 1: Modified Release Guaifenesin Suspension

### (a) Preparation of Modified Release Granules

TABLE 1
Composition of guaifenesin modified release granules

| Ingredients | mg |
|---|---|
| Guaifenesin | 600 |
| Copovidone, USP | 30 |
| Polyvinyl acetate/polyvinylpyrrolidone (Kollidon ® SR) | 70 |
| Distilled Water | q.s |

### Procedure:

Guaifenesin and polyvinyl acetate/polyvinylpyrrolidone were co-sifted and homogenously blended. The blend was granulated using aqueous solution of copovidone. Wet granules were screened, dried and sized.

### (b) Preparation of Suspension Base

TABLE 2
Composition of suspension base

| Ingredients | % w/v |
|---|---|
| Guaifenesin | 3.75 |
| Pharma grade sugar | 15 |
| Methylparaben, USPNF | 0.1 |
| Propylparaben, USPNF | 0.01 |
| Disodium EDTA, USPNF | 0.005 |
| Guar gum, USPNF | 0.80 |
| Xanthan gum, USPNF | 0.40 |
| Sodium carboxymethyl cellulose, USPNF | 0.20 |
| High fructose corn syrup, USPNF | 3 |
| Glycerol, USPNF | 3 |
| FD & C Yellow | 0.02 |
| Natural orange flavor | 0.18 |
| Polyoxyethylene sorbitan fatty acid esters, USPNF | 0.1 |
| Citric acid, USPNF | 0.25 |
| Purified water | q.s to 100 |

### Procedure:

Methylparaben, propylparaben, disodium EDTA were dissolved in purified water at 85-90° C. Pharma grade sugar was added to above solution under stirring and solution was subsequently cooled to room temperature. Guaifenesin was dissolved in 75 ml purified water and added to this sugar syrup. Guar gum, xanthan gum and sodium carboxymethyl cellulose were dispersed in high fructose corn syrup under stirring. Glycerol and citric acid was added to this dispersion. This dispersion was then added to the above sugar syrup under stirring to get uniform dispersion. Solution of color and flavor were then added.

### (c) Preparation of Modified Release Suspension

### Procedure:

4.375 g of modified release granules of guaifenesin were uniformly mixed with 60 ml of suspension base by gradual addition under stirring. This guaifenesin suspension was palatable and had the desired modified release profile.

### Example 2: Modified Release Guaifenesin Suspension

### (a) Preparation of Modified Release Granules

TABLE 3
Composition of guaifenesin modified release granules

| Ingredients | mg |
|---|---|
| Guaifenesin | 600 |
| Polyvinyl acetate/polyvinylpyrrolidone (Kollidon ® SR) | 40 |
| Sucrose stearate (HLB 3) | 40 |
| Copovidone, USP | 30 |

### Procedure:

Guaifenesin, sucrose ester and polyvinyl acetate/polyvinylpyrrolidone were co-sifted and homogenously blended. The blend was granulated using aqueous solution of copovidone. Wet granules were screened, dried and sized.

### (b) Preparation of Suspension Base

TABLE 4
Composition of suspension base

| Ingredients | % w/v |
|---|---|
| Guaifenesin | 3.75 |
| Pharma grade sugar | 15 |
| Methylparaben, USPNF | 0.1 |
| Propylparaben, USPNF | 0.01 |
| Disodium EDTA, USPNF | 0.005 |
| Guar gum, USPNF | 0.80 |
| Xanthan gum, USPNF | 0.40 |
| Sodium carboxymethyl cellulose, USPNF | 0.20 |
| High fructose corn syrup, USPNF | 3 |
| Glycerol, USPNF | 3 |
| FD & C Yellow | 0.02 |
| Natural orange flavor | 0.18 |
| Polyoxyethylenesorbitan fatty acid esters, USPNF | 0.1 |
| Citric acid, USPNF | 0.25 |
| Purified water | q.s to 100 |

### Procedure:

Methylparaben, propylparaben, disodium EDTA were dissolved in purified water at 85-90° C. Pharma grade sugar was added to above solution under stirring and solution was subsequently cooled to room temperature. Guaifenesin was dissolved in 75 ml purified water and added to this sugar syrup. Guar gum, xanthan gum and sodium carboxymethyl cellulose were dispersed in high fructose corn syrup under stirring. Glycerol and citric acid was added to this dispersion. This dispersion was then added to the above sugar syrup under stirring to get uniform dispersion. Solution of color and flavor were then added.

### (c) Preparation of Modified Release Suspension

### Procedure:

4.4375 g of modified release granules of guaifenesin were uniformly mixed with 60 ml of suspension base by gradual addition under stirring. This guaifenesin suspension was palatable and had desired modified release profile.

### Example 3: Modified Release Metformin Suspension

### (a) Preparation of Modified Release Granules

TABLE 5
Composition of metformin modified release granules

| Ingredients | mg |
|---|---|
| Metformin hydrochloride | 500 |
| Sucrose stearate (HLB 3) | 50 |
| Sucrose stearate (HLB 2) | 50 |
| Polyvinyl acetate/polyvinylpyrrolidone (Kollidon ® SR) | 50 |
| PovidneK90, USP | 30 |

### Procedure:

Metformin hydrochloride, sucrose esters and polyvinyl acetate/polyvinylpyrrolidone were co-sifted and homogenously blended. The blend was granulated using aqueous solution of povidone K90. Wet granules were screened, dried and sized.

### (b) Preparation of Suspension Base

TABLE 6
Composition of suspension base

| Ingredients | % w/v |
|---|---|
| Metformin hydrochloride | 3.75 |
| Pharma grade sugar | 15 |
| Methylparaben, USPNF | 0.1 |
| Propylparaben, USPNF | 0.01 |
| Disodium EDTA, USPNF | 0.005 |
| Guar gum, USPNF | 0.80 |
| Xanthan gum, USPNF | 0.40 |
| Sodium carboxymethyl cellulose, USPNF | 0.20 |
| High fructose corn syrup, USPNF | 6 |
| FD & C Yellow | 0.02 |
| Natural orange flavor | 0.18 |
| Polyoxyethylene sorbitan fatty acid esters, USPNF | 0.1 |
| Citric acid, USPNF | 0.25 |
| Purified water | q.s to 100 |

### Procedure:

Methylparaben, propylparaben, disodium EDTA were dissolved in purified water at 85-90° C. Pharma grade sugar was added to above solution under stirring and solution was subsequently cooled to room temperature. Metformin hydrochloride was dissolved in 75 ml purified water and added to this sugar syrup. Guar gum, xanthan gum and sodium carboxymethyl cellulose were dispersed in high fructose corn syrup under stirring. Citric acid was also added. This dispersion was added to the above sugar syrup under stirring to get uniform dispersion. Solution of color and flavor were then added.

### (c) Preparation of Modified Release Suspension

### Procedure:

4.76 g of modified release granules of metformin hydrochloride mixed with 40 ml of suspension base. This metformin suspension was palatable and has a desired modified release profile which remains the same throughout the shelf life.

### Example 4: Modified Release Memantine-Donepezil Suspension

### (a) Preparation of Modified Release Granules of Memantine Hydrochloride

TABLE 7
Composition of memantine hydrochloride
modified release granules

| Ingredients | mg |
|---|---|
| Memantine hydrochloride | 100 |
| Copovidone, USP | 15 |
| Polyvinyl acetate/polyvinylpyrrolidone (Kollidon ® SR) | 40 |
| Isopropyl alcohol | q.s |

### Procedure:

Memantine hydrochloride and polyvinyl acetate/polyvinylpyrrolidone were co-sifted through sieve #40. The mixture was granulated using copovidone dispersed in isopropyl alcohol. Wet granules were screened and dried. The dried granules were sized.

### (b) Preparation of Suspension Base

TABLE 8
Composition of suspension base

| Ingredients | % w/v |
|---|---|
| Donepezil hydrochloride | 0.1 |
| Memantine hydrochloride | 0.084 |
| Pharma grade sugar | 74 |
| Methylparaben, USPNF | 0.1 |
| Propylparaben, USPNF | 0.01 |
| Disodium EDTA, USPNF | 0.005 |
| Guar gum, USPNF | 0.80 |
| Xanthan gum, USPNF | 0.40 |
| Sodium carboxymethyl cellulose, USPNF | 0.20 |
| High fructose corn syrup, USPNF | 6 |
| FD & C Yellow | 0.02 |
| Natural orange flavor | 0.18 |
| Polyoxyethylenesorbitan fatty acid esters, USPNF | 0.1 |
| Citric acid, USPNF | 0.25 |
| Purified water | q.s to 100 |

### Procedure:

Methylparaben, propylparaben, disodium EDTA were dissolved in purified water at 85-90° C. Pharma grade sugar was added to above solution under stirring and solution was subsequently cooled to room temperature. Donepezil hydrochloride and memantine hydrochlide were dissolved in 12.5 ml purified water and added to this sugar syrup. Guar gum, xanthan gum and sodium carboxymethyl cellulose were dispersed in high fructose corn syrup under stirring. Citric acid was also added. This dispersion was then added to the above sugar syrup under stirring to get uniform dispersion. Solution of color and flavor were then added.

### (c) Preparation of Modified Release Suspension

### Procedure:

0.3038 g of modified release granules of memantine hydrochloride were added to 100 ml of suspension base. This memantine-donepezil suspension was palatable and has a desired modified release profile.

### Example 5: Modified Release Composition of Oxcarbazepine (Dry Granules-Ready for Reconstitution)

### (a) Preparation of Modified Release Granules of Oxcarbazepine

TABLE 9
Composition of oxcarbazepine modified release granules

| Ingredients | mg |
|---|---|
| Oxcarbazepine | 600 |
| Povidone, USP | 30 |
| Polyvinyl acetate/polyvinylpyrrolidone (Kollidon ® SR) | 75 |
| Distilled Water | q.s |

### Procedure:

Oxcarbazepine and polyvinyl acetate/polyvinylpyrrolidone were co-sifted and homogenously blended. The blend was granulated using aqueous solution of povidone. Wet granules were screened, dried and sized.

### (b) Preparation of Immediate Release Granules of Oxcarbazepine

TABLE 10
Composition of oxcarbazepine immediate release granules

| Ingredients | mg |
|---|---|
| Oxcarbazepine | 13.5 |
| Povidone K30, USP | 225 |
| Croscarmellose sodium, USP | 100 |
| Crospovidone, USP | 100 |
| Sodium lauryl sulphate, USP | 9 |
| Guar gum, USPNF | 360 |
| Xanthan gum, USPNF | 180 |
| Sodium carboxymethyl cellulose, USPNF | 90 |
| Disodium EDTA, USPNF | 2.25 |
| Sodium benzoate USPNF | 22.5 |
| Pharma grade sugar | 6750 |
| Polyoxyethylenesorbitan fatty acid esters, USPNF | 45 |
| Citric acid, USPNF | 112.5 |
| FD & C Yellow | 81 |
| Natural orange flavor | 9 |
| Colloidal silicon dioxide, USP | 45 |
| Distilled water | q.s |

### Procedure:

Binder solution of povidone K30 and polyoxyethylene sorbitan fatty acid esters was prepared. Oxcarbazepine and all other excipients except color, flavor and colloidal silicon dioxide were co-sifted and homogenously blended together. This blend was granulated using the above binder solution. Wet granules were screened, dried and sized. Sized granules were blended with color, flavor and colloidal silicon dioxide.

### (c) Preparation of Modified Release Formulation

### Procedure:

4.214 g of modified release oxcarbazepine granules and 8.145 g of immediate release oxcarbazepine granules were dispensed in separate sachets. Prior to administration immediate release oxcarbazepine granules were added to 45 ml of water and shaken vigorously for 10 min. Modified release oxcarbazepine granules were added to this dispersion and shaken slowly.

This oxcarbazepine suspension was palatable and had the desired modified release profile.

We claim:

1) An oral modified release composition comprising plurality of modified release matrix particles and at least one pharmaceutically acceptable excipient; wherein the modified release matrix particles comprise at least one active agent, at least one release controlling agent and at least one pharmaceutically acceptable excipient, and the modified release composition is in the form of a suspension, dry formulation for reconstitution or dispersible tablet.

2) The composition of claim 1, wherein the active agent is a psychostimulant, an antihistamine, an expectorant or a mucolytic, an anti-tussive agent, a serotonin and norepinephrine reuptake inhibitor, a sympatholytic, an antipsychotic, an antimuscarinic and a urinary antispasmodic, a PDE5 inhibitor, an anti-Alzheimer's agent, an analgesic, a decongestant, an analeptic agent, an anesthetic agent, an anti-asthmatic, an anti-arthritic agent, an anti-cancer agent, an anti-cholinergic agent, an anti-convulsant agent, an anti-depressant agent, an antidiabetic, an anti-helminthic agent, an anti-diarrheal agent, an anti-epileptic, an anti-hyperlipidemic agent, an antihypertensive, an antihypotensive, an anti-infective agent, an anti-inflammatory agent, a non-steroidal anti-inflammatory agent, an anti-emetic, an anti-migraine agent, an anti-neoplastic agent, an anti-tubercular agent, an antibiotic, an antacid, an antiulcer agent, an anti-Parkinsonism drug, an anti-pruritic agent, an antipsychotic agent, an anti-pyretic agent, an anti-spasmodic, an anti-viral agent, an anxiolytic agent, an appetite suppressant, an attention deficit hyperactivity disorder, a cardiovascular agent, a calcium channel blocker, an antianginal agent, a central nervous system agent, a beta-blocker, an antiarrhythmic agent, a bronchodilator, a central nervous system stimulant, a diuretic, a genetic material, a hormonolytics; a hypnotic, a hypercalcemic, a hypoglycemic agent, a immunosuppressive agent, a beta-agonist, a narcotic antagonist, nicotine, a nutritional agent, a parasympatholytic, a peptide drug, an antihemorrhoidal, a psychostimulant, a psychotropic, a mucolytic, a sedative, a laxative, a vitamin, a sialagogue, a steroid, a sympathomimetic, a tranquilizer, a vasodilator, or any combination thereof.

3) The composition of claim 2, wherein the active agent is in the form of free base, free acid, pharmaceutically acceptable salt, prodrug, active metabolite, polymorph, solvate, hydrate, enantiomer, optical isomer, tautomer or racemic mixture thereof, or any combination thereof.

4) The composition of claim 2, wherein the active agent is amphetamine, amphetaminil, atomoxetine, dexmethylphenidate, dextroamphetamine, dextromethamphetamine, fencamfamine, fenethylline, lisdexamfetamine, methylphenidate, mesocarb, pemoline, pipradrol, prolintane, dimenhydrinate, diphenhydramine, chlorpheniramine, brompheniramine, dexchlorpheniramine, hydroxyzine, dexbrompheniramine, fexofenadine, terfenadine, cetirizine, levocetirizine, ambroxol, bromhexine, carbocisteine, domiodol, guaifenesin, codeine, dextromethorphan, hydrocodone, clovoxamine, desvenlafaxine, duloxetine, levomilnacipran, eclanamine, milnacipran, sibutramine, venlafaxine, alaproclate, citalopram, escitalopram, femoxetine, fluoxetine, fluvoxamine, indalpine, ifoxetine litoxetine, omiloxetine, panuramine, paroxetine, pirandamine, seproxetine, sertraline, zimelidine, clonidine, guanfacine, methyldopa, iloperidone, ocaperidone, paliperidone, risperidone, lurasidone, perospirone, revospirone, tiospirone, ziprasidone, darifenacin, emepronium, fesoterodine, flavoxate, imidafenacin, meladrazine, mirabegron, oxybutynin, propiverine, solifenacin, terodiline, tolterodine, trospium chloride, acetildenafil, aildenafil, avanafil, icariin, lodenafil, mirodenafil, nitrosoprodenafil, sildenafil, sulfoaildenafil, tadalafil, udenafil, vardenafil, memantine, neramexane (1,3,3,5,5-pentamethylcyclohexan-1-amine), donepezil, tacrine, rivastigmine, galantamine, physostigmine, neostigmine, Huperzine A, icopezil (5,7-dihydro-3-[2-[1-(phenylmethyl)-4-piperidinyl]ethyl]-6H-pyrrolo-[4,5f]-1,2-benzisoxazol-6-onemaleate), ER-127528 (4-[(5,6-dimethoxy-2-fluoro-1-indanon)-2-yl]methyl-1-(3-fluorobenzyl) piperidine hydrochloride), zanapezil (TAK-147; 3-[1-(phenylmethyl)piperidin-4-yl]-1-(2,3,4,5-tetrahydro-1H-1-benzazepin-8-yl)-1-propane fumarate), metrifonate ((−)-R-α-[[2-(dimethylamino)ethoxy]methyl]benzo[b]thiophene-5-methanol hydrochloride), FK-960 (N-(4-acetyl-1-piperazinyl)-p-fluorobenzamide-hydrate), TCH-346 (N-methyl-N-2-pyropinyldibenz[b,f] oxepine-10-methanamine), SDZ-220-581 ((S)-α-amino-5-(phosphonomethyl)-[1,1′-biphenyl]-3-propionic acid), tarenflurbil, tramiprosate, clioquinol, aspirin, codeine, morphine, dihydromorphone, oxycodone, hydrocodone, phenylephrine, pseudoephedrine, theophylline, phenobarbital sodium, phenytoin sodium, valproate sodium barbiturates, amylobarbitone sodium, butabarbital sodium, secobarbital sodium, metformin; phenytoin, meprobamate, nitrezepam, oxcarbazepine, clonidine, methyldopa; captopril, propranolol, clonidine, naproxen, diclofenac, indomethacin, ibuprofen, sulindac, meclofenamate sodium, tolmetin sodium, metoclopramide, tetracyclines, atropine, scopolamine, albuterol, ethacrynic acid, bendrofluazide, nifedipine, papaverine, diltiazem, or nicardirine.

5) The composition of claim 4 wherein the active agent is guaifenesin, memantine hydrochloride, metformin hydrochloride or oxcarbazepine.

6) The composition of claim 1, wherein the release controlling agent is non-swelling polymeric release controlling agent, non-swelling non-polymeric release controlling agent or any combination thereof.

7) The composition of claim 6, wherein the non-swelling polymeric release controlling agent is a cellulose derivative, an acrylic acid derivative, a maleic acid derivative, polymer and copolymer, a vinyl derivative, polymer and copolymer or any combination thereof.

8) The composition of claim 7, wherein the non-swelling polymeric release controlling agent is ethyl cellulose, hydroxypropylmethylcellulose acetate succinate, hydroxypropylmethyl cellulose phthalate, hydroxymethylethylcellulose phthalate, cellulose acetate phthalate, cellulose acetate succinate, cellulose acetate maleate, cellulose acetate trimelliate, cellulose benzoate phthalate, cellulose propionate phthalate, methyl cellulose phthalate, carboxymethylethylcellulose, ethylhydroxyethylcellulose phthalate, styrene/acrylic acid copolymer, methyl acrylate/methacrylic acid copolymer, butyl acrylate/styrene/acrylic acid copolymer, methacrylic acid polymers and copolymers, polyacrylates, methacrylate polymers and copolymers, copolymer formed from monomers selected from methacrylic acid, methacrylic acid esters, acrylic acid and acrylic acid esters, copolymer formed from monomers selected from butyl methacrylate, (2-dimethylaminoethyl)methacrylate and methyl methacrylate, copolymer formed from monomers selected from ethyl acrylate, methyl methacrylate and trimethylammonioethyl methacrylate chloride, copolymers of acrylate and methacrylates with/without quarternary ammonium group in combination with sodium carboxymethylcellulose, dimethylaminoethyl methacrylate copolymer, trimethylammonioethyl methacrylate copolymer, ethylacrylate methylmethacrylate copolymer, methacrylic acid-methyl methacrylate copolymer, methacrylic acid-ethyl acrylate copolymer, vinylacetate/maleic acid anhydride copolymer, styrene/maleic acid anhydride copolymer, styrene/maleic acid monoester copolymer, vinylmethylether/maleic acid anhydride copolymer, ethylene/maleic acid anhydride copolymer, vinylbutylether/maleic acid anhydride copolymer, acrylonitrile/methyl acrylate/maleic acid anhydride copolymer, butyl acrylate/styrene maleic acid anhydride copolymer, polyvinylacetate, copolymers of vinyl pyrrolidone, polyvinyl alcohol, copolymers of polyvinyl alcohol, mixture of polyvinyl acetate and polyvinylpyrrolidone, Kollidon® SR, a mixture of 8 parts w/w of polyvinyl acetate and 2 parts w/w of polyvinyl pyrrolidone, polyvinyl alcohol phthalate, polyvinylacetal phthalate, polyvinyl butylate phthalate, polyvinylacetoacetal phthalate, or any combination thereof.

9) The composition of claim 6, wherein the non-swelling non-polymeric release controlling agent is sucrose fatty acid ester, glyceryl fatty acid ester, fatty acid alkyl ester, propylene glycol ester of fatty acids, sorbitan fatty acid ester, or any combination thereof.

10) The composition of claim 9, wherein the non-swelling non-polymeric release controlling agent is sucrose stearate, sucrose distearate, sucrose polystearate, sucrose palmitate, sucrose monopalmitate, sucrose dipalmitate, sucrose oleate, sucrose monooleate, sucrose dioleate, sucrose laurate, sucrose monolaurate, sucrose dilaurate, sucrose behenate, sucrose erucate, sucrose caprate, sucrose monocaprate, sucrose dicaprate, sucrose caprylate, sucrose monocaprylate, sucrose dicaprylate, sucrose myristate, sucrose monomyristate, sucrose dimyristate, sucrose linolenate, sucrose monolinolenate, sucrose dilinolenate, sucrose esters of mixed fatty acids, sucrose oligoesters, glyceryl behenate; glyceryl monostearate, glyceryl tripalmitate, glyceryl monopalmitate, glyceryl distearate, glyceryl tristearate glyceryl palmitostearate, glyceryl dipalmitate, glyceryl monoleate, glyceryl monolaurate, glyceryl dilaurate, glyceryl trilaurate, glyceryl didocosanoate glyceryl tridocosanoate, glyceryl monodocosanoate, glyceryl monocaprote, glyceryl dicaproate, glyceryl tricaproate, glyceryl monomyristate, glyceryl dimyristate, glyceryl trimyristate, glyceryl monodecenoate, glyceryl didecenoate, glyceryl tridecenoate; GLYCOWAX-932; lauroyl macrogol-32 glycerides; stearoyl macrogol-32 glyceride, glyceryl monoarachidonate, glyceryl monolinoleate, glyceryl monolinolenate, glyceryl monopalmitoleate, glyceryl monocaprylate, glyceryl monococoate, glyceryl monocollagenate, glyceryl monoerucate, glyceryl monohydroxystearate, glyceryl monoisopalmitate, glyceryl monolinoleate, glyceryl monolinolenate, glyceryl monomyristate, glyceryl monopentadecanoate, glyceryl monopolyacrylate, glyceryl monotallowate, glyceryl monothiopropionate, glyceryl monocundecylenate, isopropyl monoarachidonate, isopropyl monolaurate, isopropyl monolinoleate, isopropyl monolinolenate, isopropyl monomyristate, isopropyl monopalmitoleate, isopropyl monooleate, and isopropyl monostearate; methyl monoarachidonate, methyl monolaurate, methyl monolinoleate, methyl monolinolenate, methyl monomyristate, methyl monopalmitoleate, methyl monooleate, methyl monostearate, propylene glycol monoarachidonate, propylene glycol monolaurate, propylene glycol monolinoleate, propylene glycol monolinolenate, propylene glycol monomyristate, propylene glycol monopalmitoleate, propylene glycol monooleate, propylene glycol monostearate, propylene glycol monoarachidonate, propylene glycol monolaurate, propylene glycol monolinoleate, propylene glycol monolinolenate, propylene glycol monomyristate, propylene glycol monopalmitoleate, propylene glycol monooleate, propylene glycol monostearate, sorbitan monolaurate, sorbitan monopalmitate, sorbitan monooleate, sorbitan monostearate, sorbitan tristearate, sorbitan trioleate, sorbitan sesquioleate, sorbitan tistearate, sorbitan monoisostearate, sorbitan sesquistearate, sorbitan caprylate, sorbitan cocoate, sorbitan diisostearate, sorbitan dioleate, sorbitan distearate, sorbitan isostearate, sorbitan olivate, sorbitan sesquiisostearate, sorbitan sesquistearate, and sorbitan triisostearate or any combination thereof.

11) The composition of claim 6 wherein the release controlling agent is sucrose fatty acid ester, mixture of polyvinyl acetate and polyvinylpyrrolidone, or any combinations thereof.

12) The composition of claim 1 wherein the pharmaceutically acceptable excipient is a binder, diluent, solubility modifier, disintegrant, super disintegrant, flavor, sweetener, colorant, salivating agent, surfactant, souring agent, viscolizer, solubilizer, stabilizer, suspending agent, preservative, cosolvent, anti-caking agent, buffer, glidant, lubricant or any combination thereof.

13) The composition of claim 1 wherein the oral modified release composition in the form of a suspension comprises the modified release matrix particles dispersed in a suspension base saturated with the active agent.

14) The composition of claim 1 wherein the oral modified release composition in the form of a dry formulation for reconstitution in addition to the modified release matrix particles, further comprises immediate release particles of the active agent and/or suspension modifying particles.

15) The composition of the claim 14 wherein the oral modified release composition in the form of a dry formulation for reconstitution, on reconstitution generates a suspension base saturated with the active agent wherein the modified release matrix particles are dispersed.

16) The composition of claim 1, wherein the modified release composition further comprises an additional active agent.

17) The composition of claim 16, wherein the additional active agent is delivered in an immediate or sustained release manner.
